(19) United States
(12) Patent Application Publication
(10) Pub. No.: US 2005/0187515 A1
(43) Pub. Date: Aug. 25, 2005

(54) Reduced size programmable drug pump

(75) Inventors: Anthony J. Varrichio, Plano, TX (US); John H. Erickson, Plano, TX (US)

Correspondence Address: FULBRIGHT & JAWORSKI, L.L.P. (ANS), 2200 ROSS AVENUE, SUITE 2800, DALLAS, TX, 75201, US

(73) Assignee: Advanced Neuromodulation Systems, Inc., Plano, TX (US)

(21) Appl. No.: 11/060,241

(22) Filed: Feb. 17, 2005

Related U.S. Application Data
(60) Provisional application No. 60/545,890, filed on Feb. 19, 2004.

(51) Int. Cl. A61M 31/00

(52) U.S. Cl. 604/67

(57) ABSTRACT

Systems and methods provide a programmable or controllable infusate delivery with minimal power consumption using controllable valves and with safe and reliable operation of the delivery system. Embodiments provide programmable control without the need for implantable power sources using multi-stable valves and/or mono-stable valves which are powered externally when activated. Embodiments provide for very low power programmable control, such as by employing micro-electromechanical system valves and a flow restrictor array. An external program controller may be utilized to provide a user interface and which may communicate with the controllable infusate delivery system using wireless links. Internal controller circuitry may provide for flow control changes for different activities or times of day and/or in response to changes in pressure, temperature, etcetera. A safety valve configuration may be implemented which provides a safety flow valve configuration which responds in an opposite manner to particular events than does a corresponding primary flow valve.

### CROSS REFERENCE TO RELATED APPLICATION

The present application claims the benefit of priority to co-pending and commonly assigned U.S. Provisional Patent Application No. 60/545,890 entitled “Reduced Size Programmable Drug Pump” filed Feb. 19, 2004, the disclosure of which is hereby incorporated herein by reference. The present application is related to co-pending and commonly assigned U.S. patent application Ser. No. 10/626,902 entitled “Non-Constant Pressure Infusion Pump,” filed Jul. 25, 2003, Ser. No. 10/334,404 entitled “Apparatus for Dosage Control,” filed Dec. 30, 2002, Ser. No. 10/331,403 entitled “Method for Manipulating Dosage Control Apparatus,” filed Dec. 30, 2002, Ser. No. 10/331,425 entitled “Dosage Control Apparatus,” filed Dec. 30, 2002, Ser. No. 10/331,517 entitled “Programmable Dose Control Module,” filed Dec. 30, 2002, Ser. No. 10/755,985 entitled “Actuation System and Method for an Implantable Infusion Pump,” filed Jan. 13, 2004, and Ser. No. 10/756,673 entitled “Multi-Stable Valves for Medical Applications and Methods for Use Thereof,” filed Jan. 13, 2004, the disclosures of all of which are hereby incorporated herein by reference.

### TECHNICAL FIELD

The invention relates generally to implantable infusate delivery systems and, more particularly, to techniques for providing programmable or adjustable delivery of infusate using an implantable pump.

### BACKGROUND OF THE INVENTION

Infusate delivery pumps which are implantable in the human body are known in the art. U.S. Pat. No. 3,731,681 entitled “Implantable Infusion Pump,” U.S. Pat. No. 4,692,147 entitled “Drug Administration Device,” and U.S. Pat. No. 4,772,263 entitled “Spring Driven Infusion Pump,” the disclosures of which are incorporated herein by reference, provide several different examples of infusate delivery systems implementing different infusate propulsion mechanisms. Specifically, U.S. Pat. No. 3,731,681 provides an example of a gas driven infusate propulsion system wherein a volatile liquid partially filling one chamber provides a relatively constant pressure to an infusate chamber to expel the infusate from the pump. U.S. Pat. No. 4,692,147 provides an example of an electric motor driven infusate propulsion system wherein a peristaltic pump expels the infusate from the pump. U.S. Pat. No. 4,772,263 provides an example of a spring driven infusate propulsion system wherein a spring diaphragm provides a relatively constant pressure to an infusate chamber to expel the infusate from the pump. The aforementioned gas driven and spring driven infusate delivery pumps are often referred to as constant pressure pumps.

The foregoing examples of infusate delivery pumps, although providing acceptable performance in many situations, are not without disadvantage. For example, gas and spring driven infusate delivery pumps are typically considered non-programmable or constant rate delivery systems because the infusate propulsion system is typically set at manufacture or time of implantation (e.g., selecting an amount of volatile liquid or a vapor point of the volatile liquid for a gas driven infusate delivery pump or selecting a fluid resistance at the output of a spring driven infusate delivery pump). Although often providing programmable delivery control, such as through control of the speed of the pump motor, electric motor driven infusate delivery pumps require substantial power, requiring relatively frequent maintenance (perhaps including subsequent surgeries to expose the pump for battery replacement).

Attempts have been made at providing an infusate delivery pump providing a controllable delivery control with low power consumption. For example, U.S. Pat. No. 6,048,328 entitled “Implantable Drug Infusion Device Having an Improved Valve,” the disclosure of which is incorporated herein by reference, provides a multi-stable valve configuration for controlling infusate delivery rate. This type of pump has been referred to as a programmable fixed rate pump, and are generally limited to a few selectable flow rates which are fixed until reprogrammed.

While an infusate delivery pump which uses latchable valves generally requires little or no power to stay open or closed, power is required to change states. It is important in such a device to confirm any change of state and to assure that the device remains in a selected state. For example, a multi-stable valve may fail to latch into a selected state in response to a control signal, necessitating a sensing network to be implemented for confirming a desired change in state. Additionally, a multi-stable valve may spontaneously or undesirably change states, such as in response to a physical shock, an external electric or magnetic field, a change in pressure, etcetera, necessitating a sensing network to be implemented for confirming a desired state. Typically, such programmable fixed rate pumps have required more valves and electronics and are more limited in their capabilities and delivery rate reliability than the currently available electric motor driven infusate delivery pumps, although exhibiting improved power performance.

### BRIEF SUMMARY OF THE INVENTION

The present invention is directed to systems and methods which provide programmable or controllable infusate delivery with minimal power consumption using controllable valves in a configuration which provides safe and reliable operation of the delivery system. Embodiments provide an infusate delivery system (also referred to herein as an infusate pump or drug pump) in which infusate delivery rates may be programmed or controlled after implantation in a human body, and most preferably throughout the life of the system, such as to deliver a bolus, to provide a different prescription for different activities or times of day, etcetera.

Embodiments of the present invention provide programmable control without the need for implantable power sources. For example, using multi-stable valves which consume no power to hold a selected state, embodiments of the invention utilize an external programmer to change states of a valve or valves, providing the power for such a state change through RF or other means. Mono-stable valves which, although consuming appreciable power to change state and to be held in that state, may similarly be used, such as to deliver a bolus, without requiring an internal power supply by powering these valves by external means.

Alternative embodiments of the present invention provide for very low power programmable control. For example, one embodiment uses a watch-like circuit, which can store two or more times of day and two or more dosages which the pump automatically and/or via patient activation can change dosages. Alternative embodiments utilize a multiple stage battery configuration to provide long battery life while providing relatively high current delivery throughout the useful life of the infusate pump.

Control of infusate delivery rates may be in response to control manipulation, such as by a physician or patient, or may be automated. For example, an actuator may be provided for altering the state of a multi-stable valve or valves to provide control of an infusate delivery rate. Similarly, a wireless link, such as implementing radio frequency (RF), magnetic, or capacitave coupled communication, may be utilized to control altering the state of a valve or valves to provide control of an infusate delivery rate. Additionally or alternatively, a control system may be implemented to provide automated control for altering the state of a valve or valves. According to one embodiment, a control system is utilized to provide automated control for compensation for infusate flow rate associated with the nonlinearity of the infusate reservoir pressure and/or temperature. Similarly, a control system may be implemented to provide automated control of an infusate flow rate to create dosages which lie between actual fixed choices by varying the duty cycle at each setting.

Embodiments of the invention utilize automated control of infusate delivery rates improve infusate delivery accuracy. For example, a reservoir calibration constant may be stored electronically within the pump, such as in a control system thereof, to calibrate infusate delivery to the particulars of the infusate reservoir propulsion mechanism (e.g., the spring drive system of a particular infusion pump). Similarly, calibration data may be utilized in providing for correction for the nonlinearity of the reservoir propulsion mechanism. For example, the reservoir pressure exerted by a spring diaphragm may vary from the mean by approximately +/−2%, resulting in the amount of infusate delivered on the tenth day after a reservoir being filled being as much as 24% less than that delivered on the thirtieth day, for a forty day drug protocol. However, this behavior is predictable and can be electronically corrected using the aforementioned calibration data and knowing the reservoir contents or knowing when the reservoir is full and calculating contents based on the known programmed flow rate. Reservoir level monitoring may be accomplished, for example, using capacitive or Hall Effect detection circuitry.

Because assuring latched valves maintain their position will likely be important to the operation of an infusate delivery pump system in many situations, embodiments of the present invention implement various valve monitoring features, such as may periodically compare valve positions to program settings and/or which can control a redundant safety valve in the event of a valve position error. Monitoring valve position may be done in various ways according to the present invention, depending on valve type and material. For example, valve position may be monitored by measuring the conductivity or capacitance of a lever arm to a pinch plate. Additionally or alternatively, valve position may be monitored by sensing optical or electromagnetic interference in one position (latch state). Embodiments of the invention use a Hall Effect device to sense valve position.

Preferred embodiments of the invention are adapted to provide a fail safe and/or safety valve architecture. For example, primary control valves and corresponding safety valves may be disposed in reversed configurations and/or positions to mitigate unlatching problems due to shock, vibration or magnetic fields. That is, a primary and safety valve configuration is implemented according to embodiments of the invention such that an event that causes a primary control valve to spontaneously open will cause a corresponding safety valve to close, thereby preventing an undesired flow of infusate.

Infusate delivery systems of embodiments of the present invention preferably provide for wireless communication of information, such as using RF, capacitive coupling, magnetic field, etcetera. Preferred embodiments provide for far field (e.g., 6 to 30 ft) communication from an implanted infusate delivery system. Such communications may be used to inform/alarm a patient that they are near or at a condition upon which action must be taken, such as the infusate reservoir is empty or that a power supply is in need of recharging. Additionally or alternatively, such communications may be utilized for changing/monitoring the infusate delivery system operational status.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWING

For a more complete understanding of the present invention, reference is now made to the following descriptions taken in conjunction with the accompanying drawing, in which:

FIG. 1 shows a prior art infusate pump configuration;

FIG. 2 shows an infusate pump configuration according to embodiments of the present invention;

FIGS. 3A and 3B show detail with respect to an infusate delivery control block of an infusate pump according to embodiments of the present invention; and

FIG. 4 shows detail with respect to an infusate delivery control block of an infusate pump according to an embodiment of the present invention.

### DETAILED DESCRIPTION OF THE INVENTION

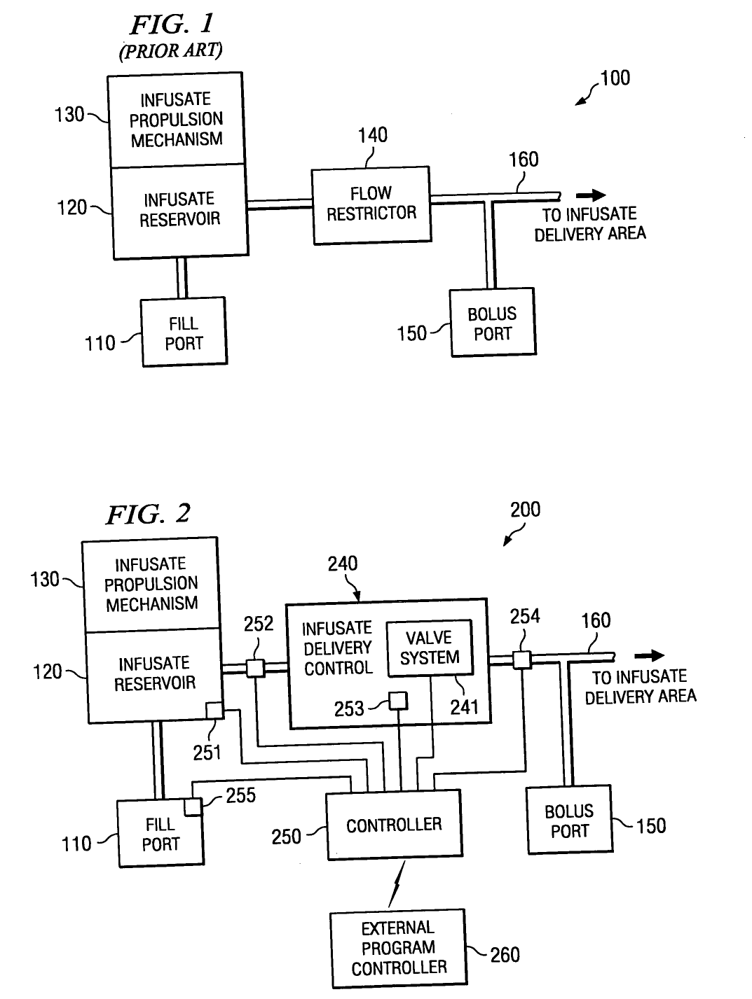

Directing attention to FIG. 1, a high level block diagram of atypical constant pressure infusate pump, such as may be implanted in a human body to dispense a drug or other pharmacological agent to a portion of the body over time, is shown as infusate pump 100. Infusate pump 100 includes infusate reservoir 120 which stores an amount of fluid containing a drug or other pharmacological agent prescribed to a patient for treatment. Fill port 110, such as may comprise a needle septum, is in fluid communication with infusate reservoir 120 to facilitate introduction of fluid into infusate reservoir 120. Infusate propulsion mechanism 130, such as may comprise a gas or spring diaphragm, provides a relatively constant pressure to infusate reservoir 120 to expel the infusate from infusate pump 100. Infusate which is expelled from infusate reservoir 120 passes through flow restrictor 140, such as may comprise a fluid conduit of restricted diameter and/or media to resist the flow of fluid, which controls a rate at which the infusate escapes infusate pump 100. Flow restrictor 140 is in fluid communication with pump output 160, such as may be coupled to a catheter routed to a portion of the body requiring treatment, to provide a desired flow of infusate from infusate pump 100. Bolus port 150, such as may comprise a needle septum, is also in fluid communication with pump output 160 to facilitate delivery of a bolus to a portion of the body requiring treatment.

The above described infusate pump is a relatively simple and effective way to provide delivery of infusate at a substantially constant rate. However, the pump configuration does not easily allow for alteration of an amount of infusate delivered. For example, infusate pump 100 does not readily provide for programming to deliver a different prescription, such as at various times of the day or in response to particular activities.

Directing attention to FIG. 2, infusate pump 200 adapted according to embodiments of the present invention is shown in a high level block diagram. Infusate pump 200 of the illustrated embodiment shares several functional aspects with infusate pump 100 discussed above and, therefore, those functional blocks share the same reference numerals.

Infusate pump 200 includes infusate delivery control 240 and controller 250 operatively coupled to provide programmable or adjustable delivery of infusate according to embodiments of the invention. Infusate delivery control 240 of the illustrated embodiment includes valve system 241, which may comprise one or more controllable valves, operative under control of controller 250 to control delivery of infusate by infusate pump 200. Infusate delivery control 240 of various embodiments may include additional fluid control aspects, such as one or more flow restrictors (e.g., a fluid conduit of restricted diameter and/or media to resist the flow of fluid). It should be appreciated that the above mentioned valves and flow restrictors may be provided as separate components (e.g., a discrete valve and discrete flow restrictor) or as integrated components (e.g., a valve providing flow restrictor functionality) and may be disposed in any relationship with respect to one another in the flow path according to embodiments of the invention.

Controller 250 may comprise a processor based system operating under control of an instruction set defining operation as described herein. For example, controller 250 may comprise an application specific integrated circuit (ASIC). Valves of valve system 241 may comprise one or more micro-electromechanical system (MEMS) valve, piezeo-electric valve, magnetic valve, solenoid valve, constriction valve, and/or the like. Controller 250 and infusate delivery control 240 preferably provide for efficient power usage during operation. Accordingly, controller 250 may comprise a watch-like circuit which can store two or more times of day and two or more dosages which the pump automatically and/or via patient activation can change dosages. Such a watch-like circuit may be provided power periodically, such as by RF signals or through kinetic energy conversion circuits. Additionally or alternatively, controller 250 may become dormant or substantially dormant during extended periods of time, such as by being responsive to an external programmer to change states of a valve or valves, with the power for such a state change provided through RF or other means. Valves of valve system 241 may comprise multi-stable valves which require application of power only when a change of state is desired.

Infusate pump 200 of the illustrated embodiment includes sensors 251-255 disposed at various locations within infusate pump 200 and which are operatively coupled to controller 250 to provide information thereto. Sensors 251-255 may each comprise one or more sensory collectors. For example, sensor 253 may collect infusate temperature information, infusate pressure information, and valve state information to be used by controller 250. The number and placement of sensors may be different than illustrated for alternative embodiments of the invention.

According to a preferred embodiment, sensors may be disposed and/or configured to provide information with respect to a plurality of aspects of infusate pump operation. For example, sensor 251, which may provide infusate reservoir volume information, may be utilized to detect a leak in one or more valves of valve system 241 through analysis of a desired flow rate and the rate at which the infusate reservoir volume is changing. Additionally, sensor 251 may be utilized to provide feedback with respect to a doctor filling or refilling infusate reservoir 120, such as through RF communication with external program controller 260. For example, if a doctor has inserted a needle into the patient to refill infusate reservoir 120 and sensor 251 does not reflect an increase in volume in infusate reservoir 120 as the doctor injects the infusate, fill port 110 has not been properly engaged by the needle. Similarly, if a refill operation attempts to overfill infusate reservoir 120, such as by overextending a spring diaphragm of infusate propulsion mechanism 130, sensor 251 may detect the condition. External program controller 260 may report the foregoing conditions to the doctor using information from sensor 251 communicated by RF or other wireless means by controller 250 to external program controller 260.

It should be appreciated that sensory information provided by one or more of sensors 251-255 may provide information with respect to position or proximity of various elements. For example, Hall effect devices, capacitive coupling, inductance coils, optical detectors, etcetera may be utilized in determining the position of a valve element and, thus, the state of the valve. Likewise, such position or proximity detection apparatus may be utilized in determining the current volume of infusate reservoir 120, such as by disposing a plate upon a spring diaphragm of infusate propulsion mechanism 130 and having a corresponding plate or Hall effect detector disposed within infusate reservoir 120. According to one embodiment, inductive coils are disposed within fill port 110 (shown as sensor 255 in FIG. 2) to detect when a needle penetrates the septum thereof. In such an embodiment, the metal of the needle may be relied upon to affect the inductance of the coil, and thereby provide confirmation that fill port 110 has been accessed by the refill needle. A similar sensor may be utilized with respect to bolus port 150, if desired. Irrespective of whether a sensor is used in both fill port 110 and bolus port 150, information regarding whether the correct septum has been accessed by a needle delivering infusate may be provided according to embodiments of the invention.

Various mechanisms may be utilized in combination with or in place of one or more of the above mentioned sensors to provide desired operation of infusate pump 200. For example, in addition to sensor 251 providing information with respect to infusate reservoir 120 being filled with infusate, a mechanical check valve may be implemented between infusate reservoir 120 and fill port 110, such that when infusate reservoir 120 reaches a full state the check valve cuts off the fluid communication between fill port 110 and infusate reservoir 120 to prevent overfilling. A doctor refilling infusate pump 200 may be informed of the full condition by information from sensor 251 being presented by external program controller 260 and/or by a sudden increase in injection pressure felt in the syringe used in the refill operation.

Infusate pump 200 and external program controller 260 preferably cooperate to provide far field (e.g., 1 to 30 feet) wireless communications (e.g., RF, capacitive coupled, magnetic field, etcetera). According to one embodiment, external program controller 260 comprises a processor based system having memory and input/output apparatus to provide a user interface with respect to infusate pump 200 to a doctor and/or patient. For example, a doctor may utilize a keyboard, pointer, and/or voice interface of external program controller 260 to establish settings for providing a desired infusate delivery rate which is then communicated wirelessly to controller 250 in order to set states of valves of valve system 241. Additionally or alternatively, program controller 260 may provide a means by which a bolus may be administered (whether by a doctor or a patient), such as by manipulating a button or speaking an appropriate command. Of course, one or more such functions may be provided security, such as by requiring an appropriate access code be entered into external program controller 260 and/or infusate pump 200 or by limiting the times and/or periods at which such functions may be performed. Similarly, the wireless link between infusate pump 200 and external program controller 260 may be secure, such as through use of encryption techniques well known in the art.

The user interface provided by embodiments of external program controller 260 may additionally or alternatively provide information to users, such as a doctor or patient. For example, external program controller 260 may be worn on the person of a patient or kept in a same room as the patient to provide real-time status information with respect to infusate pump 200. For example, external program controller 260 may present information with respect to the volume of infusate remaining within infusate reservoir 120 on a graphical display of external program controller 260. Additionally or alternatively, external program controller 260 may provide an audible alarms upon detection of particular conditions, such as when the volume of infusate remaining within infusate reservoir 120 becomes critically low, when infusate reservoir 120 is being overfilled, when a flow valve malfunctions, when a battery is becoming depleted, etcetera.

In operation according to one embodiment, infusate flow is controlled by infusate pump 200 by a flow restrictor of infusate delivery control 240 and controlling a flow valve of valve system 241. Such a configuration may utilize a mono-stable valve, such as a solenoid valve, which is normally closed and using power to maintain an open state. The flow valve may be controlled on and off (open and closed) to modulate a given flow rate as provided by the restrictor. A control valve duty cycle can be selected to give an average infusate flow rate which corresponds to a desired rate. For example, a flow rate suitable for delivering pain blocking pharmaceuticals to the spine using infusate pump 200 may cycle the flow valve open for a few milliseconds (e.g., 30 milliseconds) periodically every few seconds or minutes (e.g., every 75 seconds) to provide a very small time averaged flow rate.

It should be appreciated that, when a desired infusate flow rate is at or near that provided by the flow restrictor in the above embodiment, a mono-stable valve configuration would be open for a long period of time in order to provide a desired flow rate. Accordingly, the flow restrictor may be selected to provide a flow rate greater than that typically desired in operation, such as a maximum safe infusate flow rate. Such a configuration facilitates power consumption economy by using short “on” or open pulses of a mono-stable valve to deliver a desired flow rate. Alternatively, a multi-stable valve configuration may be utilized according to embodiments of the invention. Multi-stable valves may be utilized to provide a flow valve that draws zero power when open and closed, thereby facilitating use of a more restrictive flow restrictor and holding the flow valve in an open position longer without utilizing excessive amounts of power.

A configuration in which the aforementioned flow restrictor provides a flow rate greater than that desired provides risks in the event the aforementioned flow valve becomes stuck in the open state. Accordingly, embodiments of the invention provide a configuration in which valve system 241 includes multiple valves in series to provide redundancy. Using redundant valves, if one valve fails, a second valve should remain operative. However, experience has shown that a simple redundantancy system is insufficient to provide reliable operation as there is nothing to indicate the failure of the first valve and, thus, use continues with only the redundant valve operating. Such use with only the redundant valve functioning is essentially a non-redundant system which suffers from the risks discussed above.

Accordingly, embodiments of the present invention are adapted to detect valve malfunctions and thus ensure safe operation with a redundant valve configuration. According to one embodiment, pressure differentials with respect to various portions of the fluid flow path are monitored to detect flow valve malfunction. For example, closing the flow valve on the low pressure side of a redundant valve configuration last (i.e., closing the flow valve on the high pressure side of the redundant valve configuration first) and measuring the pressure in the fluid flow path between these two flow valves, such as using sensor 253) if the flow valve on the high pressure side is leaking an increase in pressure should be detected. Conversely, closing the flow valve on the high pressure side of a redundant valve configuration last (i.e., closing the flow valve on the low pressure side of the redundant valve configuration first) and measuring the pressure in the fluid flow path between these two flow valves, if the flow valve on the low pressure side is leaking a decrease in pressure should be detected. Both of the foregoing flow valve closing techniques and pressuring monitoring may be implemented periodically to detect the proper operation of the infusate pump system.

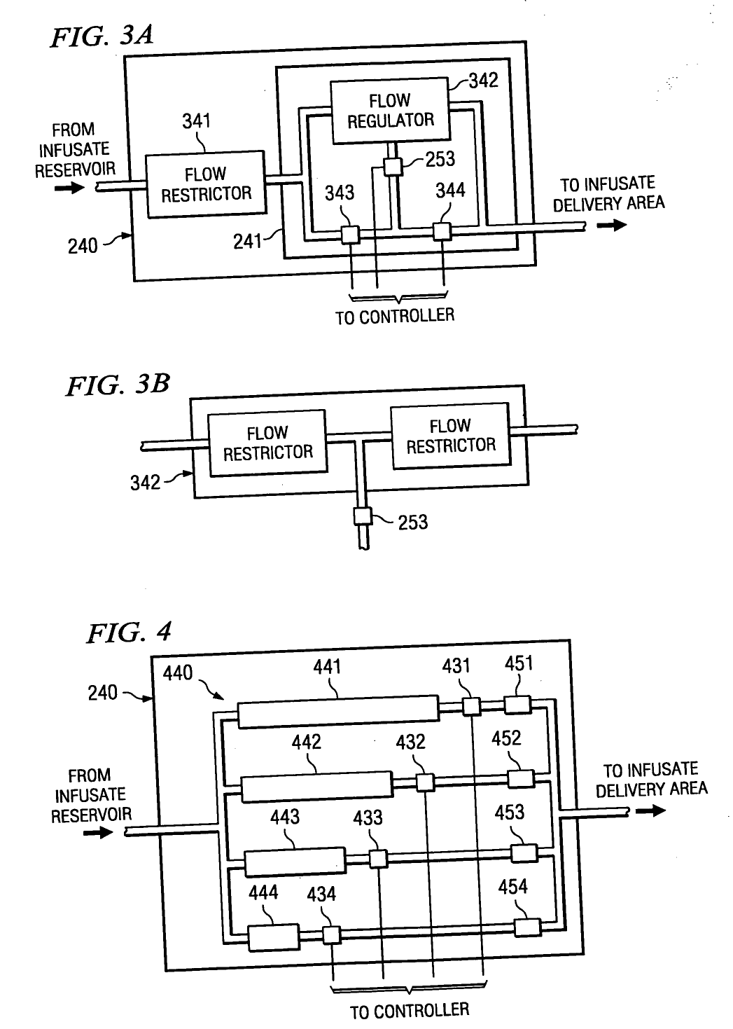

FIG. 3A shows another embodiment adapted to detect malfunction of a flow valve in a redundant valve configuration. In the embodiment illustrated in FIG. 3A, valve system 241 includes primary flow valve 343 disposed on a high pressure side and redundant flow valve 344 disposed on a low pressure side. In normal operation both primary flow valve 343 and redundant flow valve 344 are controlled to open and close in cycles to deliver a desired time averaged flow rate to a patient. However, the embodiment of FIG. 3A also includes a fluid flow path bypassing primary flow valve 343 and redundant flow valve 344, that includes flow restrictor 342. Flow restrictor 342, since it allows fluid flow to bypass fluid valves 343 and 344, preferably provides a minimum desired flow rate. A center tap is provided from flow restrictor 342 (as may be implemented by flow restrictor 342 comprising two flow restrictor portions as shown in FIG. 3B)c and the junction between primary flow valve 343 and redundant flow valve 344 for use in detecting malfunction of either flow valve. For example, if the midpoint pressure, as may be sensed by sensor 253, neither flow valve is leaking. However, if one of the flow valves leaks, this midpoint pressure will drift (higher if primary flow valve 343 is leaking and lower if redundant flow valve 344 is leaking).

The embodiment illustrated in FIG. 3A includes optional flow restrictor 341. Flow restrictor 341 may provide a relatively high flow rate, such as a maximum safe flow rate (e.g., 1250 milliliters of infusate a day), to facilitate acceptably short “on” or open cycles of flow valves 343 and 344 while providing a safe, although perhaps undesired, flow rate in the event of failure of the redundant valve system (i.e., the infusate pump is limited to a predetermined maximum flow rate by flow restrictor 341). Such a configuration may be particularly desirable where the infusate pump is to provide a bolus using the flow valves. The flow restriction provided by flow restrictor 341 may be selected to provide a flow rate sufficiently low to be safe if the valves fail open and yet provide a flow rate sufficiently high to allow the valves to be held open for the bolus without excessive energy consumption.

Experimentation has revealed that a multi-stage battery configuration may be utilized to implement the foregoing redundant valve configuration which efficiently provides extended operation of the valves, even where valves 343 and 344 are mono-stable normally closed solenoid valves, for time averaged delivery of a desired flow rate as well as allowing periodic bolus flows. Specifically, a primary battery may be implemented which provides suitable energy over an extended period of time (e.g., 8-10 years), wherein a secondary battery or batteries, charged by the primary battery, may be utilized to deliver short bursts of sufficient power to hold valves open for a sufficient duration to deliver a bolus.

Such a configuration is preferred according to an embodiment of the present invention because batteries that have the ability to deliver high current pulses throughout their entire life have a tendency to self-discharge. However, it is desirable to have an extended life expectancy of an implantable device, such as the above described infusate pump, to minimize trauma and inconvenience to a patient. Batteries that reliably deliver power throughout an extended lifetime tend to be, or become, high impedance, limiting their ability to deliver high current pulses. The foregoing multi-stage battery configuration addresses these issues and provides an energy source which is both long lived and is capable of delivering high current pulses throughout a reasonable service life for an infusate pump.

According to one embodiment, a primary battery is provided using a power cell, such as a lithium-iodine cell, as is commonly used in pacemakers and other implantable electronic devices today. However, such batteries, although generally providing power for extended periods of time, tend to become very high impedance with time, preventing their delivering relatively large bursts of energy. Accordingly, secondary batteries having different impedance characteristics may be utilized to provide bursts of energy suitable for opening flow valves and holding flow valves open for a bolus. Such secondary batteries may be rechargeable lithium-ion rechargeable cells, such as may be in the form of thin disks which are similar in appearance to flat ceramic capacitors.

In operation, using some filtering and perhaps a voltage multiplier, the primary battery may be utilized to open and/or close valves, whereas the secondary batteries may be utilized to provide the energy to hold the valve state for a period of time. For example, the primary battery may be coupled to a voltage doubler to provide sufficient voltage to open a selected valve, and perhaps hold the valve open for some relatively small amount of time (e.g., 30 milliseconds). The primary battery may therefore be used to pulse valves 343 and 344 to provide a controlled amount of infusate delivered. If the valve is to be held open for a period of time longer than is supported by the primary battery circuit (e.g., 1-3 seconds for a bolus), a secondary battery capable of sustaining relatively large currents for such a burst may be coupled to the valve. The primary battery may be utilized to trickle charge the secondary batteries between bursts. Such a configuration has been found to be suitable to facilitate holding a valve, such as a normally closed solenoid valve, open for a few seconds at a time, thus facilitating delivery of a bolus even with the safety of flow restrictor 341 in the fluid flow path.

It should be appreciated that in the embodiment illustrated in FIG. 3A, the fluid resistance provided by flow restrictors 341 and 342 are known and the time that flow valves 343 and 344 are opened may be controlled. Although the pressure delivered to infusate reservoir 120 by infusate propulsion mechanism 130 is relatively constant, some variation in this pressure is typical. For example, a spring diaphragm may be calibrated to provide a preselected pressure midpoint (e.g., 8 psi), but may provide pressures varying around this midpoint (e.g., 7%) depending upon the extent of spring extension at the time. Accordingly, the pressure provided in expelling the infusate may be at least slightly variable.

Controller 250 of one embodiment may obtain infusate pressure information from a sensor or sensors, such as sensors 251 and 252, and adjust a period of open cycles used with respect to flow valves 343 and 344 to result in a desired time average amount of infusate being delivered to the infusate delivery area.

Variations in the pressure provided by an infusate propulsion system may be characterized, such as at time of manufacture. This pressure characterization information may be utilized in controlling flow valve cycles without continuous infusate pressure measurement information. For example, a memory of controller 250 may store infusate propulsion system pressure characterization information and utilize this information and knowledge of when infusate reservoir 120 was last filled in adjusting the duration of open cycles of flow valves 343 and 344 in correspondence with the pressure of infusate then being expelled from infusate reservoir 120.

Embodiments of the present invention additionally or alternatively utilize multi-stable valve configurations to provide a configuration having efficient energy consumption characteristics. For example, multi-stable MEMS valve structures, e.g., nano-scale electrostatic gate valves, may be utilized which require application of energy for a change of state, but which maintain a selected state with little or no applied energy. Other embodiments of multi-stable valves may be utilized, such as mechanically latching valve mechanisms, according to embodiments of the present invention. However, MEMS valve structures may be preferred according to embodiments of the present invention as 10-20 micron filter assemblies may be formed in the MEMS substrate itself, eliminating handling, cleaning, and assembling discrete filters as are commonly used in infusate pumps.

Multi-stable valves of embodiments of the invention may be used in combination with the above mentioned mono-stable valves. For example, mono-stable solenoid valves may be utilized as described above to provide a controllable bolus with minimal energy requirements while multi-stable valves are utilized to provide a programmable continuous flow rate for delivery of a prescription.

In one embodiment, an external program controller may be utilized to control adjustment of multi-stable valves to deliver a predetermined prescription, allowing controller 250 of infusate pump 200 to monitor and maintain the multi-stable valve states, and to pulse the mono-stable valves to deliver a bolus. Accordingly, such an external program controller may provide the relatively large amount of energy utilized by the mono-stable valves, such as through RF transmission, without calling upon the internal power supply of the infusate pump for this operation. Such an embodiment provides extremely safe operation in that the infusate pump may be configured such that it is not capable of delivering a bolus without the external program controller. Such an external controller may be programmed to allow a bolus only under certain conditions, certain times, and/or under control of certain individuals (e.g., a doctor or a nurse using a proper access code). Moreover, infusate pumps may be provided without an internal power supply, relying upon an external program controller to both set multi-stable valves for delivery of a continuous flow rate and/or to control delivery of a bolus.

According to one embodiment of the invention, a flow restrictor array is utilized in combination with controllable valves, e.g., the aforementioned multi-stable valves, to facilitate a programmable flow rate. Directing attention to FIG. 4, flow restrictor array 440, comprising at least a part of infusate delivery control 240, is shown. Although not shown in the illustration of FIG. 4, restrictor array 440 may be utilized in combination with other infusate delivery control apparatus. For example, restrictor array 440 may comprise flow restrictor 342 of FIG. 3A, thereby providing a combination in which flow valves 343 and 344 provide a bolus and restrictor array 440 provides programmable continuous infusate delivery.

The illustrated embodiment of restrictor array 440 provides a binary ladder configuration to facilitate programmable infusate delivery rates. Specifically, flow restrictor 441, operable under control of flow valve 431, provides a highest flow rate (e.g., 1.2 milliliters per day), flow restrictor 442, operable under control of flow valve 432, provides approximately 1/z the highest flow rate (e.g., 0.6 milliliters per day), flow restrictor 443, operable under control of flow valve 433, provides approximately V4 the highest flow rate (e.g., 0.3 milliliters per day), and flow restrictor 444 provides approximately 1/8 the highest flow rate (e.g., 0.15 milliliters per day). Although the embodiment shown provides a 4 bit binary ladder, embodiments of the present invention may utilize any number of bits in such a ladder. However, it is envisioned that there will be a finite limit of ratios that can be effectively resolved using a binary ladder approach. Accordingly, one or more bits may be controlled (e.g., pulsed) to provide a time average approximation of a lesser flow rate. For example, pulse control of flow valve 434 may be provided such that this least significant bit (LSB) effectively operates as the last four bits of a 7 bit restrictor array.

In operation, a desired infusate flow rate may be selected by opening select ones of flow valves 431-434, while leaving closed the remaining ones of valves 431-434, for which the associated flow restrictors sum to provide the desired flow rate. Control of flow valves 431-434 is preferably provided by control signals from controller 250. Where a flow rate is desired which does not correspond to that provided by a flow restrictor of flow restrictors 441-444 or a combination thereof, one or more of flow valves 431-434 may be periodically cycled (pulsed) to provide a time averaged flow rate corresponding to the desired flow rate.

Because embodiments of flow valves 431-434 maybe susceptible to accidental or unexpected changes of state in certain situations, e.g., MEMS devices may be very small and susceptible to a change in state due to an overpressure condition, a shock, exposure to a large magnetic field, etcetera, embodiments of the present invention implement a safety valve configuration. For example, flow valves 451-454 are shown disposed in each of the binary ladder flow paths to provide a safety valve configuration. In a preferred embodiment, flow valves 451-454 are comprised of a same valve structure as are corresponding ones of flow valves 431-434. However, flow valves 451-454 are preferably disposed and/or configured to provide an opposite change of state in response to an stimulus resulting in an undesired change of state to a corresponding one of flow valves 431-434. For example, where flow valve 431 comprises a gate valve susceptible to “blowing” open in an overpressure condition, flow valve 451 may comprise a gate valve disposed in a reversed orientation with respect to the flow path to thereby “blow” closed in the same overpressure condition. The flow valves providing a safety valve configuration may similarly be disposed and/or configured to provide opposite changes in states in response to such stimulus as shock, magnetic fields, etcetera. For example, where a primary flow valve is normally closed and an electrostatic charge is used to hold the valve open, a corresponding safety flow valve may be comprised of a normally open valve for which an electrostatic charge is used to hold the valve closed, thereby providing an opposite reaction to stimulus likely to cause an undesired change in state with respect to the primary flow valve.

Although shown as including one safety valve in each flow path, it should be appreciated that a combination of safety valves may be utilized, if desired. For example, a safety valve may be provided in a flow path to respond to a overpressure condition, another safety valve may be provided in the same flow path to respond to a physical shock, and still another safety valve may be provided in the same flow path to respond to a magnetic field.

It should be appreciated that separate safety valves need not be provided for each flow path of the binary ladder of FIG. 4. For example, one or more safety valves may be disposed in a portion of the flow path shared by one or more of the binary ladder components, thereby providing a safety valve for multiple primary flow valves. However, an embodiment wherein individual safety valves are provided with respect to different flow paths may be preferred in order to facilitate continued operation (although perhaps not optimal operation) even in the event of an event causing one or more safety valves to engage.

The foregoing safety valve configurations may be utilized according to embodiments of the present invention to safely provide a flow path which bypasses the restrictor array, such as for delivering a bolus. For example, rather than using a mono-stable valve configuration, as discussed with respect to FIG. 3A, a multi-stable valve configuration which provides more efficient energy consumption attributes, although perhaps providing a less resilient valve mechanism, may be utilized. According to one embodiment, valves 343 and 344 of FIG. 3A are multi-stable valve mechanisms, such as the aforementioned MEMS devices, arranged in a safety valve configuration as described above, to provide a very low power bolus delivery system.

Although not shown in the illustration of FIG. 4 for simplicity, it should be appreciated that flow valves 451-454 may be coupled to a controller to provide control with respect to a state thereof. Moreover, one or more sensors, such as sensor 253 may be provided in restrictor array 440, such as to monitor a state of flow valves 431-434 and/or flow valves 451-454, to monitor infusate pressure into, in, and/or out of restrictor array 440, etcetera. For example, Hall effect sensors or capacitive coupling may be utilized to determine the position of electrostaticly operated valves.

In addition to or in the alternative to using sensors to detect the state of a flow valve, embodiments of the present invention may operate to periodically provide a control signal to select the desired state with respect to one or more valves. For example, where flow valve 431 and 433 are programmed to be in an open state and flow valves 432 and 434 are programmed to be in a closed state, controller 250 may periodically provide a valve open control signal to flow valves 431 and 433 and a valve close control signal to flow valves 432 and 434 to ensure that the valves are kept in the desired states. Similarly, controller 250 may periodically provide a valve open control signal to safety flow valves 451-454, perhaps after determining that primary flow valves 431-434 are in their desired states.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. An implantable infusate pump comprising:
an array of flow restrictors disposed in a plurality of fluid flow paths; and
a plurality of controllable valves, wherein valves of said plurality of controllable valves are disposed in different ones of said fluid flow paths.

2. The infusate pump of claim 1, wherein said array of flow restrictors comprise a binary ladder configuration.

3. The infusate pump of claim 1, wherein said plurality of controllable valves comprise multi-stable valves.

4. The infusate pump of claim 3, wherein said multi-stable valves comprise micro-electromechanical system (MEMS) devices.

5. The infusate pump of claim 4, wherein a substrate of said MEMS devices provides an infusate filter.

6. The infusate pump of claim 3, wherein said plurality of controllable valves comprise at least one mono-stable valve.

7. The infusate pump of claim 6, wherein said at least one mono-stable valve is disposed in a fluid flow path which bypasses said array of flow restrictors.

8. The infusate pump of claim 6, wherein said at least one mono-stable valve provides a controllable bolus valve.

9. The infusate pump of claim 6, wherein said mono-stable valve is provided power to change state from an external source via a wireless link.

10. The infusate pump of claim 9, wherein at least one valve of said multi-stable valves is provided power to change state from a source internal to said infusate pump.

11. The infusate pump of claim 1, further comprising:
a controller coupled to said plurality of valves and providing control signals thereto to select valve states.

12. The infusate pump of claim 11, further comprising:
at least one sensor coupled to said controller.

13. The infusate pump of claim 12, wherein said at least one sensor detects insertion of a needle into a fill port septum.

14. The infusate pump of claim 12, wherein said at least one sensor detects a volume of infusate in an infusate reservoir.

15. The infusate pump of claim 12, wherein said at least one sensor detects a state of a valve of said plurality of valves.

16. The infusate pump of claim 11, wherein said controller includes a wireless communication interface adapted to provide far field wireless communication with an external program controller.

17. A method for delivering infusate, said method comprising:
providing an array of flow restrictors disposed in a plurality of fluid flow paths of an infusate pump;
providing a plurality of controllable valves disposed in different ones of said fluid flow paths; and
controlling said controllable valves to provide a desired aggregate infusate flow through a predetermined combination of one or more flow restrictor of said array of flow restrictors.

18. The method of claim 17, wherein said providing said array of flow restrictors comprises providing said array of flow restrictors in a binary ladder configuration.

19. The method of claim 17, wherein said controlling said controllable valves comprises changing a state of at least one valve from a first state of multi-stable valve states to a second state of said multi-stable valve states.

20. The method of claim 17, wherein said controlling said controllable valves comprises changing a state of at least one valve from a first state of mono-stable valve states to a second state of said mono-stable valve states.

21. The method of claim 17, further comprising:
providing power to alter a state of one or more said controllable valves when controlling said controllable valves to provide a desired aggregate infusate flow from an external programmer.

22. The method of claim 21, wherein said providing power comprises:
providing a radio frequency power signal.

23. The method of claim 17, further comprising:
controlling a controllable valve disposed in a fluid flow path bypassing said array of flow restrictors to deliver a bolus.

24. The method of claim 17, further comprising:
sensing a state of at least one valve of said plurality of controllable valves using a sensor coupled to a controller.

25. An implantable infusate pump comprising:
an infusate reservoir;
an infusate delivery fluid path coupled to said infusate reservoir; and
a remotely controllable bolus valve disposed in said infusate delivery fluid path at a point providing sufficient fluid flow to deliver a bolus.

26. The implantable infusate pump of claim 25, wherein said infusate delivery fluid path comprises a portion for delivering a desired flow rate over time and a portion for delivering an increased flow rate, said bolus valve being disposed in said portion for delivering said increased flow rate.

27. The implantable infusate pump of claim 26, wherein said portion for delivering a desired flow rate over time comprises a flow restrictor array.

28. The implantable infusate pump of claim 26, wherein a flow restrictor adapted to provide a maximum safe flow rate is disposed in the flow path before said bolus valve.

29. The implantable infusate pump of claim 25, further comprising:
a multi-stage battery circuit in which a primary battery stage provides power to change a state of said bolus valve and a secondary battery stage provides power to maintain said state of said bolus valve once changed.

30. The implantable infusate pump of claim 29, wherein said secondary battery state is recharged by said primary battery stage.

31. The implantable infusate pump of claim 29, wherein said primary battery stage comprises a lithium-iodine cell and said secondary battery stage comprises a lithium-ion cell.

32. The implantable infusate pump of claim 25, wherein a power source for changing a state of said bolus valve is provided externally to said infusate pump and is coupled thereto wirelessly.

33. The implantable infusate pump of claim 25, wherein said remotely controllable bolus valve is provided power to change states from an external programmer.

34. The implantable infusate pump of claim 33, wherein said remotely controllable bolus valve comprises at least one multi-stable valve.

35. The implantable infusate pump of claim 33, wherein said remotely controllable bolus valve comprises a plurality of multi-stable valves disposed in a fail safe valve architecture.

36. The implantable infusate pump of claim 35, wherein said fail safe valve architecture comprises a primary valve and a safety valve.
